(19) United States
(12) Patent Application Publication
(10) Pub. No.: US 2012/0251386 A1
(43) Pub. Date: Oct. 4, 2012

(54) HYBRID STERILIZATION

(75) Inventors: Gerald R. WOOTTON, Cambridge (CA); Catherine THACKER, Cambridge (CA)

(73) Assignee: ATS AUTOMATION TOOLING SYSTEMS INC., Cambridge (CA)

(21) Appl. No.: 13/433,404

(22) Filed: Mar. 29, 2012

Related U.S. Application Data
(60) Provisional application No. 61/470,418, filed on Mar. 31, 2011.

(51) Int. Cl. A61L 2/18; A61L 2/00

(52) U.S. Cl. 422/26; 422/292; 422/300

(57) ABSTRACT

There is described a method for sterilizing a device. The method includes: masking a portion of the device from a sterilization protocol using a mask; sterilizing a portion of the device using a first sterilization protocol; and sterilizing a portion of the device using a second sterilization protocol; where the mask shields the masked portion of the device from at least one of the sterilization protocols or attenuates the effect of at least one of the sterilization protocols in the masked portion. There is also described a system for sterilizing a device. The system includes a first sterilant; a second sterilant; and a mask for shielding a portion of the device from at least one of the sterilants.

### CROSS REFERENCE TO RELATED APPLICATIONS

This application claims the benefit of priority of U.S. Provisional Patent Application No. 61/470,418 filed Mar. 31, 2011, which is incorporated herein by reference in its entirety.

### FIELD

The present disclosure relates generally to a method and system for sterilizing products. More particularly, the present disclosure relates to a method and system for sterilizing products with components that require differing sterilization methods.

### BACKGROUND

Some devices, assemblies, parts or the like may be challenging to sterilize due to potential differences in tolerance of various components to various sterilization methods. Such devices, assemblies, parts, and the like are sometimes referred to herein as “complex devices”. For example, some medical devices that require sterilization fit into this category.

In order to sterilize the complex device, one may sterilize the many components individually, using appropriate sterilization methods for each component, and then assemble the components into the complex device in an aseptic process while maintaining the sterility of each of the components during all phases of handling and transport. In one example of an aseptic process, components or subassemblies are input already sterilized and contained in sterile packaging. Outer packaging is removed in a clean environment and then loaded into one or more interlocks. Inner packaging is removed after sterilizing the packaging and the interlock, sterilization may be the hybrid sterilization described above for assemblies or complex parts. Parts are then passed into an aseptic chamber where they are assembled. Since components or subassemblies are input to the assembly machine already sterilized, in order to avoid contamination, it is necessary to maintain sterilizing within the assembly machine for a period of productive time.

In such aseptic processes, risk to the product is low, however there is still residual risk associated with the integrity of packaging and the aseptic environment, particularly any contamination of the assembly tooling. The use of sterile packaging for parts supply and the additional sterilization in the interlock may add considerable cost and complexity. Accordingly, such a process may increase logistic complexity and increase risk to product sterility, while incurring the extra overheads of managing a complex aseptic assembly process.

It is, therefore, desirable to reduce the amount of post sterilization assembly, such as by facilitating in-line sterilization (since an in-line sterilization process reduces logistical complexity and reduces risk of product contamination). One method of reducing post sterilization assembly is to sterilize the whole complex device after it has been assembled (generally referred to as terminal sterilization).

In one example of terminal sterilization, one may immerse the device in a sterilizing chemical bath such as a reactive vapor or liquid bath. However, sterilizing chemical baths may produce product damage for some components, may have limited penetration of complex devices, may require prolonged exposure times, and/or may leave undesirable residue on surfaces. Also, some organisms may be highly resistant requiring prolonged exposure to achieve a result; particularly, lower reactivity chemicals may be used in order to minimize product damage but with consequential extended process times and/or reduced efficacy. One example of a reactive vapor which is used for terminal sterilization is ethylene oxide (EtO), which is toxic and requires a protracted and explosion-prone protocol that must be performed in a certified chamber separate from the assembly floor.

An alternative terminal sterilization method consists of exposing the finished product to penetrating radiation. However, this method also has its deficiencies including a potential decomposition of some materials, possible screening by highly absorptive materials, induced voltages or currents harmful to sensitive electronics, release of reactive ions and/or volatile compounds and hazardous secondary radiation which may sometimes be more hazardous than the primary radiation. In particular, medical devices that are functionalized by organic materials such as proteins, peptides, DNA units, etc where material selection can not be optimized for resistance to some sterilization protocols, exposure to strongly reactive chemicals and/or ionizing irradiation is not generally feasible. On the other hand, radiation is generally desirable since it is typically faster acting than many chemical methods. Also, penetration of the ionizing radiation into interior recesses of a device does not depend on the size of possible points of ingress; furthermore, sterilization may be accomplished after packaging closures and seals have been formed. Unlike other methods, fixtures, tooling carriers or other holding devices may be made to be transparent to the radiation so that these items may not limit exposure to the sterilant.

Another terminal sterilization method is exposure to non-penetrating radiation. Such a terminal sterilization method applies only to closed forms lacking in openings and rebates where radiation may be applied from multiple directions.

On the other hand, partial sterilization followed by aseptic processing is regarded as a last alternative due to the attendant risks as well as the difficulty in demonstrating the probability of a non-sterile unit (PSNU) during the processing of an entire batch of devices. However, this approach does permit various components or subassemblies to be sterilized using different methods.

Even when a terminal sterilization is used, additional sterilization of critical components and subassemblies and/or in-line sterilization during assembly may be beneficial, for example, to reduce the number of live organisms available to contaminate the assembly process or to reduce the number of organisms that must be neutered during terminal sterilization. Further, additional sterilization methods may be applied selectively to critical components to improve the effectiveness of the terminal sterilization method.

It is, therefore, desirable to provide improved systems and methods for sterilizing complex devices.

### SUMMARY

It is an object of the present disclosure to eliminate or mitigate at least one disadvantage of one or more previous sterilizing apparatuses and methods by, for example: avoiding sterilizing components separately and then combining them to form a device while maintaining the sterility of each component until they are combined; reducing the need for multiple sterilizations of separate components, aseptic assembly or intermediate sterile packaging; reducing at least one disadvantage associated with terminal sterilizations; or reinforcing equipment sterility through repeated exposure to one or more sterilization protocols.

In one aspect, the present disclosure provides a method for sterilizing a device. The method includes: masking a portion of the device from a sterilization protocol using a mask; sterilizing a portion of the device using a first sterilization protocol; and sterilizing a portion of the device using a second sterilization protocol; where the mask shields the masked portion of the device from at least one of the sterilization protocols or attenuates the effect of at least one of the sterilization protocols in the masked portion.

The mask may alter the first sterilization protocol, the second sterilization protocol, or both the first and the second sterilization protocols so that shielding the masked portion of the device results in exposure of the masked portion to an altered sterilization protocol.

In another aspect, the present disclosure provides a method for sterilizing at least a portion of a device. The method includes: masking a first portion of the device from a first sterilization protocol using a first mask, resulting in a first unmasked portion of the device; sterilizing the first unmasked portion of the device using the first sterilization protocol; and sterilizing the device using a second sterilization protocol.

The method may further include: masking a second portion of the device from a second sterilization protocol using a second mask, resulting in a second unmasked portion of the device; where the step of sterilizing the device using a second sterilization protocol comprises sterilizing the second unmasked portion of the device using the second sterilization protocol.

The first mask may alter the first sterilization protocol, the second sterilization protocol, or both the first and the second sterilization protocols so that shielding the masked portion of the device results in exposure of the masked portion to an altered sterilization protocol.

The first mask, the second mask, or both the first and second masks may alter the first sterilization protocol, the second sterilization protocol, or both the first and the second sterilization protocols so that shielding the masked portion of the device results in exposure of the masked portion to an altered sterilization protocol.

The first mask, the second mask, or both the first and the second mask may be an item introduced into the process, or may inherently be a portion of the device.

The methods may further include removing at least one mask from the device.

The first sterilizing protocol and the second sterilizing protocol may be applied sequentially or concurrently.

One or more portions of the device may be exposed to both the first and the second sterilization protocols.

The methods may further include masking a portion of the device with a further mask and sterilizing the unmasked portion of the device using a further sterilization protocol.

In yet another aspect, the disclosure provides a system for sterilizing a device. The system includes: a first sterilant; a second sterilant; and a mask for shielding a portion of the device from at least one of the sterilants.

The mask may be a component attached to or embedded in the device to be sterilized.

The mask may be a component that is removed when the device is used.

The system may further include another mask for shielding a portion of the device from the other of the first and second sterilants, or from a further sterilant.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIGS. 1A-C are flow charts illustrating exemplary methods according to aspects herein;

FIG. 2 is an illustration of a system according to another aspect herein;

FIG. 3 is an illustration of a system according to another aspect herein;

FIG. 4 is an illustration of a system according to another aspect herein; and

FIG. 5 is a flow chart illustrating an assembly process which incorporates a method according to yet another aspect herein.

### DETAILED DESCRIPTION

Generally, the present disclosure provides a method and system for sterilizing devices, for example devices which comprise components which have differing sterilization requirements.

In one example of a method according to the present disclosure, the method includes: masking a portion of the device from a sterilization protocol using a mask; sterilizing a portion of the device using a first sterilization protocol; and sterilizing a portion of the device using a second sterilization protocol; where the mask shields the masked portion of the device from at least one of the sterilization protocols.

In another example of a method according to the present disclosure, the method includes: masking a first portion of the device from a first sterilization protocol using a first mask, resulting in a first unmasked portion of the device; sterilizing the first unmasked portion of the device using the first sterilization protocol; and sterilizing the device using a second sterilization protocol.

In an example of a system according to the present disclosure, the system includes: a first sterilant; a second sterilant; and a mask for shielding a portion of the device from at least one of the sterilants.

Shielding a portion of the device with a mask would be understood to mean that the mask attenuates or stops the effect of a sterilization protocol in the masked portion, or that the mask alters the sterilization protocol such that the masked portion is sterilized by the altered sterilization protocol.

### Sterilization Method

In an embodiment of a method for sterilizing complex devices, portions of a device are sterilized using at least one sterilization protocol while other portions of the device are sterilized by at least one protocol where the totality of sterilization protocols differs for different proportions of the device. In this method, various sterilization protocols may be applied sequentially, nearly concurrently or concurrently. In another aspect, device supports, which may be carriers, holding fixtures, grasping tools, packaging, etc, may be sequentially, nearly concurrently or concurrently sterilized.

In an alternate embodiment of the method, a portion or portions of a device are masked or shielded from at least one sterilization protocol, a portion of a device is sterilized using a first sterilization protocol and a portion of the device is sterilized using a second sterilization protocol, where the masked portion of the device is shielded from at least one of the sterilization protocols. In some cases, the portions covered by dissimilar sterilization protocols may be joined by an overlapping region such that there is no area which is not sterilized; consequently, these regions are exposed to at least two sterilization protocols. In another aspect, the mask may serve to alter rather than suppress the sterilization protocol such that the portion masked off is sterilized by the altered sterilization protocol.

In some other particular cases, a portion may be excluded from or exposed to at least one additional sterilization protocol without masking by various means such as confinement, directional radiation, negative masks, etc.

It will be apparent to one skilled in the art that, although desirable in some situations, it is not necessary for the whole of the device to be sterilized according to the method described above. A device to be sterilized could be assembled from an unsterilized portion and a previously sterilized portion, and the sterilization method described above is applied to the unsterilized portion.

Depending on the sterilization protocols being used, the sterilization protocols may be applied in sequence or concurrently. The sterilization protocols may be the same or different. In methods herein, the masks may be: a component attached to or embedded in the device to be sterilized; a part carrier or portion thereof used to hold or manipulate the device during the sterilization process; or any additional item or tooling used to provide a masking function. A component attached to or embedded in the device may be, for example, a sealed packaging, a cap, or an item that is removed at point of use. A part carrier or portion thereof used to hold or manipulate the device may be, for example, a part tray, a holding fixture, or a grasping tool. Any step of removing a mask that may be removed at point of use (for example, removing a cap) is optional.

In an exemplary method, a first portion of a device is masked from a first sterilization protocol using a first mask, resulting in a first unmasked portion of the device; the first unmasked portion of the device is sterilized using the first sterilization protocol; and the masked portion of the device is sterilized using a second sterilization protocol.

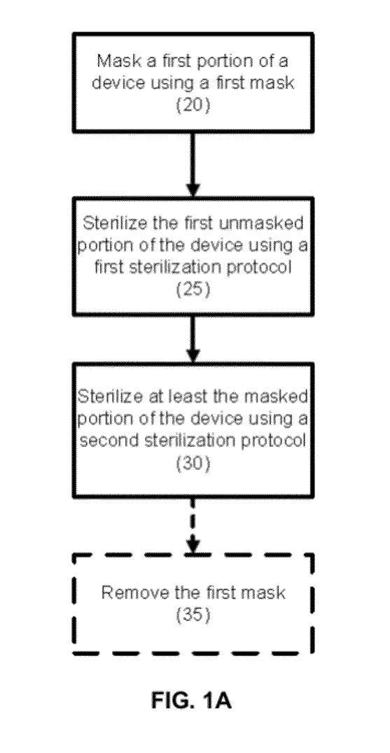

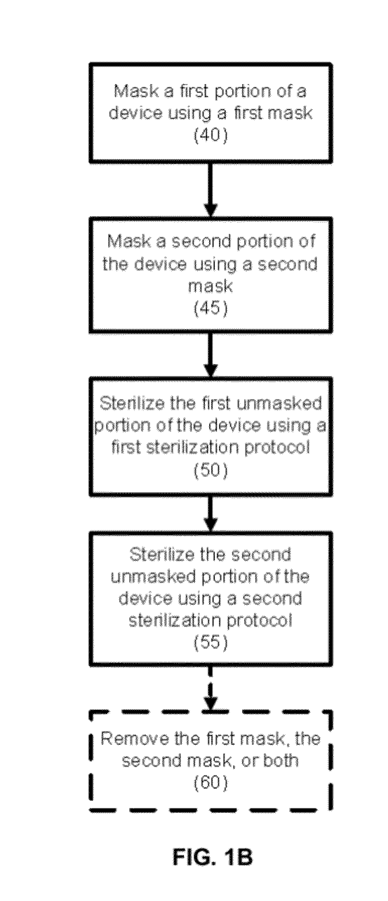

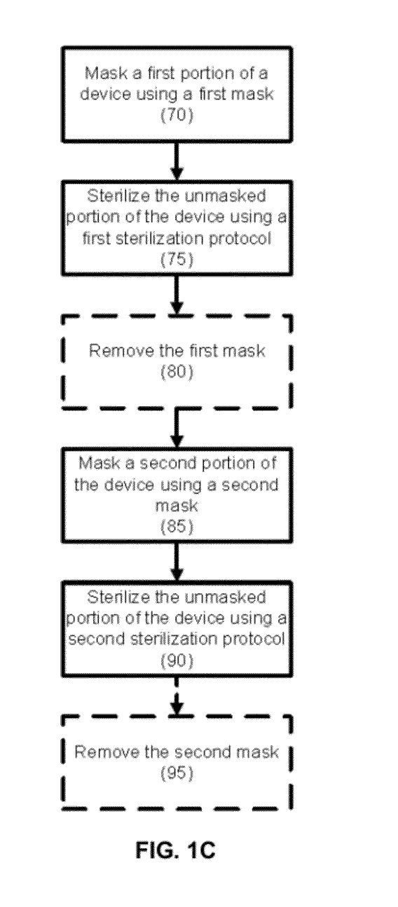

The exemplary method is illustrated in FIG. 1A where a first portion of a device is masked from a first sterilization protocol using a first mask (20), resulting in a first unmasked portion of the device; the first unmasked portion of the device is sterilized using a first sterilization protocol (25); at least the first masked portion of the device is sterilized using a second sterilization protocol which is not impeded by the first mask (30); subsequently, as illustrated by the stippled line, the first mask may optionally be removed (35). Alternatively, the first mask may be retained by the device. Additional masks and sterilization protocols may be used as desired, for example in order to achieve full coverage. In some methods, some portions of the device, for example an unmasked area near the perimeter of the first mask, may be sterilized by both the first and second sterilization protocols.

Another exemplary method according to the present application is illustrated in FIG. 1B. As illustrated, a first portion of a device is masked from a first sterilization protocol using a first mask (40), resulting in a first unmasked portion of the device; a second portion of a device is masked from a second sterilization protocol using a second mask (45), resulting in a second unmasked portion; the first unmasked portion of the device is sterilized using a first sterilization protocol (50); the second unmasked portion of the device is sterilized using a second sterilization protocol (55) where each of the sterilization protocols is not prevented by at least one of the masks. The first mask, the second mask or both the first and second masks may optionally be removed (60), as illustrated by the stippled line. Alternatively, the masks may be retained by the device. Additional masks and sterilization protocols may be used as desired, for example in order to achieve full coverage.

Another exemplary method according to the present application is illustrated in FIG. 1C. As illustrated, a first portion of a device is masked from a first sterilization protocol using a first mask, resulting in a first unmasked portion of the device (70); the first unmasked portion of the device is sterilized using the first sterilization protocol (75); the first mask may optionally be removed (80), as illustrated by the stippled line; a second portion of the device is masked from a second sterilization protocol using a second mask, resulting in a second unmasked portion of the device (85); the second unmasked portion of the device is sterilized using the second sterilization protocol (90); and the second mask may optionally be removed (95), as illustrated by the stippled line. Additional masks and sterilization protocols may be used as desired, for example in order to achieve full coverage.

Another exemplary method according to the present application is one where a portion of the device is protected by a mask, the unprotected area is sterilized by at least one sterilization protocol and the protected area is sterilized by at least one other sterilization protocol which penetrates the mask or is contained within the masking device. Preferably, the at least one other sterilization protocol sterilizes at least the entire portion covered by the mask. In this method, concurrent sterilization is possible.

Another exemplary method according to the present application is one where a portion of the device is protected by a mask that alters the sterilization protocol in a selected area. In one example, the mask absorbs at least one sterilizing radiation and emits at least one other sterilizing radiation which impinges on at least the portion of the device that is covered by the mask. Either all of the impinging radiation is blocked and the alternative radiation is substituted or only some of the impinging radiation is blocked and the alternative radiation augments the primary radiation.

Contemplated sterilization protocols include chemical sterilization methods including vapor, surface reaction vapor, plasma, reactive ion, liquid bath and hot air, possibly augmented by sonic agitation, and radiation sterilization methods including penetrating and non-penetrating radiation including gamma, beta, ion beam, E-beam, X-ray, UV, flash lamp and laser radiation. In certain combinations, sterilization protocols may augment or amplify the efficacy of at least one sterilization protocol. In others, the reverse may be true, in which case, a mask may also be used to isolate the various sterilization protocols from each other.

In methods that use chemical and radiation sterilization protocols, at least one portion of the device is masked by a masking device that provides a gas-tight seal. An exemplary method includes: the unprotected portion of the device is exposed to a chemical vapor or liquid while the protected portion of the device is exposed to radiation which penetrates through the mask. Penetrating radiation may also sterilize the masking device itself. In this method, penetrating radiation may be localized to impinge only on the mask area. Alternatively, it may impinge broadly on some or on all of the device. In this case, it may be further desirable to protect one or more portions of the device from this radiation by means of a second mask, for example, a first portion of the device is masked using a radiation shield and second portion of the device is masked using a liquid or vapor barrier. The chemical vapor or liquid sterilization protocol and the radiation sterilization protocol may be applied consecutively or concurrently. Advantageously, the interaction of the radiation and the vapor or liquid may result in the liberation of reactive ions and/or secondary radiation that may augment the chemical sterilization process.

Contemplated protocols include vapor plasma and E-beam sterilization. Vapor plasma is a multiphase method which includes chemical reaction followed by a burst of UV and a flux of reactive ions in the discharge phase which is fast and effective but not very penetrating i.e. interior cavities and deep recesses may not be effectively sterilized. This is however, a desirable method since it is very fast. On the other hand, E-Beam is highly penetrating and may sterilize the problem areas even more quickly. Interactions between the E-beam and the plasma may not be favorable; however, the E-beam may be applied before and/or after plasma discharge. Advantageously, vapor plasma is generally applied in a low pressure chamber which improves the effectiveness and precision of E-beam radiation.

In an alternative exemplary method, the device is sterilized using two penetrating radiation sterilization protocols. A first portion of the device is masked using a first radiation shield and irradiated from one axis, and a second portion of the device is masked using a second radiation shield and irradiated from a different axis. The two radiation sterilization protocols may be applied consecutively or concurrently.

Although the terms “first” and “second” are used to differentiate the masks, the portions of the device, the sterilization protocols, and other elements or steps, it will be apparent to one skilled in the art that the terms do not reflect an order of operation. In this regard, a “first sterilization protocol” could be applied after a “second sterilization protocol” is applied, and a step of “masking a first portion of the device” could be undertaken after a step of “masking a second portion of the device” is undertaken.

### Sterilization System

In a system according to the present application, there is provided a first sterilant, a second sterilant, and a mask for shielding a portion of the device from at least one of the sterilants. The system could optionally include one or more sterilants, and/or one or more masks for shielding one or more other portions of the device from one or more of the sterilants. As discussed below, the mask may be a component which is permanently, semi-permanently, or temporarily attached to, embedded in, or affixed to the device to be sterilized. Alternatively, in certain circumstances, the mask may be a tooling or fixturing that is otherwise used in the manufacturing process.

A system according to the present application could employ, for example, chemical vapor and radiation sterilization as two sterilization protocols. Such a system could include a radiation-shield (i.e. a mask) that prevented or attenuated radiation from impacting a radiation-sensitive component, while still allowing the radiation-sensitive component to be exposed to the chemical vapor sterilization protocol. Alternatively, such a system could include a vapor barrier (i.e. a mask) around a chemically-sensitive component to prevent chemical vapor from contacting the chemically-sensitive component, while still allowing the chemically-sensitive component to be exposed to the radiation sterilization protocol. In cases where it is further desirable to segregate sterilization protocols the mask may also provide this function; for example, the mask may further be integrated with a radiation or chemical vapor source to provide the necessary confinement. This is preferable as it permits concurrent application of multiple sterilization protocols where a sequential application would otherwise be needed.

Various combinations of masks and sterilization protocols could be used to selectively sterilize predetermined components using pre-selected sterilization protocols. For example, radiation (for example ionizing irradiation (e.g. UV irradiation), or penetrating radiation), and chemical sterilants (e.g. gas, vapor, plasma, and vapor/plasma chemical sterilants) could be used to sterilize the components.

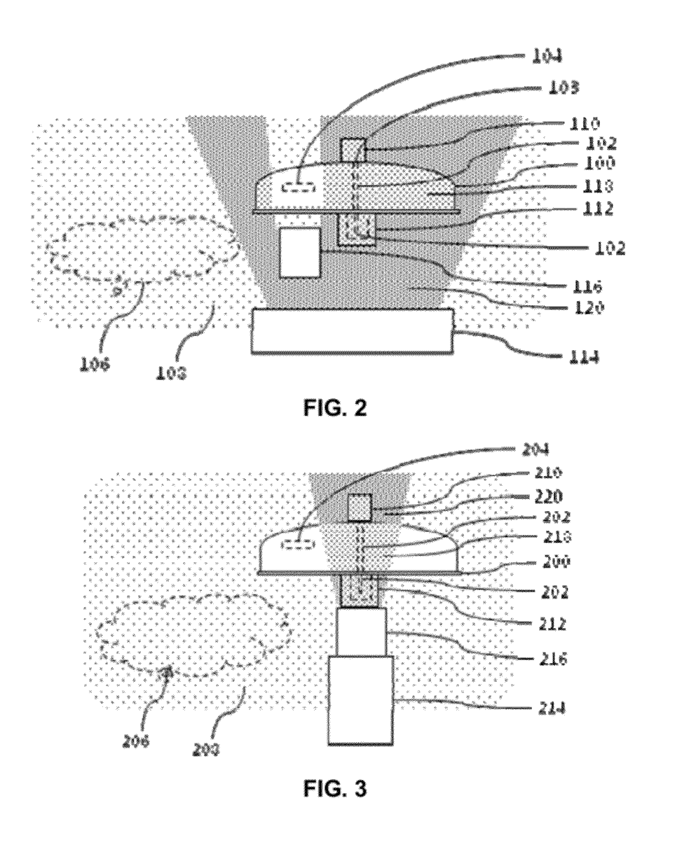

An exemplary system according to the present application is illustrated in FIG. 2, which shows a device 100 to be sterilized. The device 100 includes a portion 102 to be shielded against chemical sterilization, and a portion 104 to be shielded against penetrative radiation sterilization. The device 100 is sterilized using a chemical sterilant 106, which fills a volume 108 of the sterilization chamber. A vapor seal 110 and a cap 112 cover aperture 103 and shield (i.e. mask) the portion 102 from the chemical sterilization. Radiation source 114 provides penetrating radiation with a broad area of coverage, while radiation shield 116 shields (i.e. masks) the portion 104 from the penetrative radiation sterilization. Shaded area 118 illustrates the portion of the device 100 which is sterilized by penetrative radiation. Shaded area 120 illustrates the area covered by both chemical sterilization and penetrating radiation.

Another exemplary system according to the present application is illustrated in FIG. 3, which shows a device 200 to be sterilized. The device 200 includes a portion 202 to be shielded against chemical sterilization, and a portion 204 to be shielded against penetrative radiation sterilization. The device 200 is sterilized using a chemical sterilant 206, which fills a volume 208 of the sterilization chamber. A vapor seal 210 and a cap 212 shield (i.e. mask) the portion 202 from the chemical sterilization. Radiation source 214 and beam director 216 provide penetrating radiation with a narrow area of coverage, shielding (i.e. masking) the portion 204 from the penetrative radiation sterilization. Shaded area 218 illustrates the portion of the device 200 which is sterilized by penetrative radiation. Shaded area 220 represents an area that is sterilized both by chemical steriliant 206 and penetrating radiation source 214 and beam director 216.

In this example, a cap protects a sensitive component that protrudes from the body of the device while a seal covers an aperture that leads to the interior of the device. In this example, these masking devices could be applied temporarily, in which case, they could advantageously be elements of a holding fixture or part carrier although they may also be reusable or disposable components that protect the sensitive components of the device for at least the sterilization process and possibly other manufacturing processes. Alternatively, these masking devices could be removed at point of use or broken during use, in which case, they would provide a permanent extra layer of protection for the sensitive area.

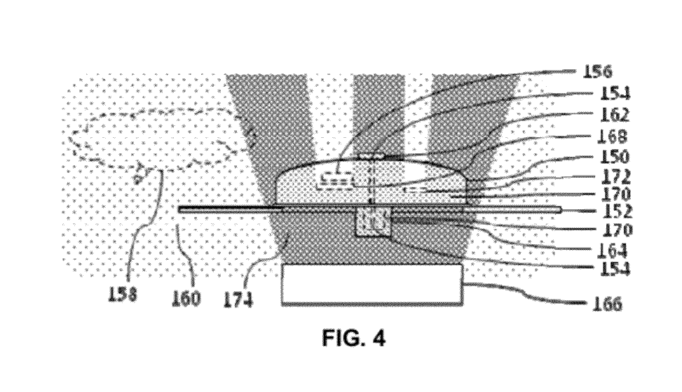

Another exemplary system according to the present application is illustrated in FIG. 4, which shows a device 150 to be sterilized. The device 150 includes a portion 154 to be shielded against chemical sterilization, and a portion 156 to be shielded against penetrative radiation sterilization. The device 150 is presented on a carrier or fixture 152, which is also sterilized in the process. The device 150 is sterilized using a chemical sterilant 158, which fills a volume 160 of the sterilization chamber. A vapor seal 162 and a cap 164 shield (i.e. mask) the portion 154 from the chemical sterilization. Radiation source 166 provides penetrating radiation. An embedded mask 168 shields (i.e. masks) the portion 156 from the penetrative radiation sterilization. Shaded area 170 illustrates the portion of the device 150 which is sterilized by penetrative radiation. Another embedded mask 172 depicts a portion of the device that may act as a mask to attenuate the penetrative radiation but is unaffected by it, however portions of the device that are masked are nevertheless sterilized by the chemical sterilant. Shaded area 174 illustrates the area surrounding the device that is sterilized by both the chemical sterilant 158 and the radiation source 166.

In this example, the cap 164 protects a sensitive component that protrudes from the body of the device while the vapor seal 162 covers an aperture that leads to the interior of the device. In this example, the vapor seal 162 may be, for example, a foil seal or membrane seal that is applied to the product prior to sterilization and is either removed, punctured or displaced at a later time, for example at the point of use, while in the mean time maintaining sterility of the enclosed portion of the device. In one example, the vapor seal is a foil seal which attenuates radiation but whose outer surface is nevertheless sterilized by the chemical sterilant 158. In this example, the cap 164 is a protective cap which protects and encapsulates a sensitive component preserving its sterility, and which may be removed later, for example at the point of use. The cap may be, for example, attached to the product in such a way to indicate tampering or other breach of protection. In this example, the device 150 is supported by a carrier or fixture 152 and at least a portion of the device 150 is also sterilized with penetrating radiation 170, ensuring that areas of contact between the device 150 and carrier 152 which may not be adequately sterilized by the chemical sterilant 158 are nevertheless sterilized. Embedded mask 172 may attenuate sterilizing radiation, but may not be harmed by it; however, exposed portions of the device 150 which are shielded by mask 172 are at least sterilized by the chemical sterilant 158 where these portions would otherwise be inadequately sterilized or would require additional exposure time.

One or more of the masks may be applied temporarily to protect sensitive components of the device for at least one of the sterilization processes and possibly other manufacturing processes. One or more of the masks may be removed at point of use or broken during use, in which case they would provide a permanent extra layer of protection for a sensitive area. Masks which are removed at point of use or broken during use, such as vapor seal 162 and/or cap 164, may provide an extra level of protection against contamination should sterile packaging containing the device be damaged, or after the sterile packaging has been removed.

### Sterilization Protocols

Radiation may be used to sterilize various products. Sterilizing radiation may include, for example, ionizing irradiation and penetrating radiation. Different sources of radiation are known in the art and may be used to irradiate the product. Example non-ionizing radiation includes narrow spectrum UV-A, UV-B, UV-C and broad spectrum UV, high-intensity broad spectrum or white light, laser and fast laser and high-intensity IR. It should be noted that UV and short pulse laser radiation is not, strictly speaking, non-ionizing. This radiation is generally non-penetrating; however, for items that are essentially transparent, it may be. Other forms of radiation include X-rays, ion-beam, E-beam, beta, gamma and other high-energy particles. These are generally considered to be penetrating radiation although absorption by some materials may be substantial and possibly limit their efficacy. Radiation is typically fast acting and may be to a great extent trimmed to the requirement i.e. by varying intensity, spatial distribution and exposure time. In some cases, radiation is produced indirectly; for instance, it is common to derive X-rays by irradiating a target with another radiation such as gamma rays.

The radiation could be provided, for example, by means of a collimator, a focusing element and/or a beam scanner. Collimated gamma radiation sources could be scanned over selected areas of the product, for example by means of a moving aperture or by means of moving the source and beam director. An E-beam could be focused and/or directed, for example by means of electromagnetic or electrostatic deflection. A beam directing element could be used to direct the angle of incidence of the beam. Close proximity of the radiation to the device, and/or use of focusing elements, may allow for closely controlled coverage of the radiation sterilization. This controlled coverage may reduce unwanted damage or other undesirable side-effects of radiation.

The radiation source may be mounted on a moving stage so that a single radiation source may be used to irradiate different portions of the product. Using a portable source of radiation (for example a radiation source located on one or more movable stages), particularly a continuous radiation source (e.g. gamma source), may allow for selective irradiation of the product, and may provide a means of moving the radiation source to a safe position during operator interventions or service of the system.

Radiation sources may provide very rapid and effective sterilization, but may damage some materials and certain types of components. For example, radiation may result in electronic devices being damaged, digital memory being erased, batteries being discharged, and/or polymers being degraded. Radiation sources may generate secondary radiation in some materials, which may be used to provide an additional mode of sterilization. Ionizing radiation may also be used to create reactive ions which have a sterilizing effect, for example within an enclosed space where the radiation is also penetrating.

Chemical sterilants may be used to sterilize various products. Common vaporous sterilants include steam, hydrogen peroxide, formic acid, ethelyne oxide, alcohol, formaldehyde, ketone, acetone. Such chemical sterilants may be injected into a sterilization chamber and allowed to sterilize any exposed surface of the product. The chemical sterilant may be a gas, vapor, a plasma, or a gas/vapor which is converted to a plasma (for example a low temperature plasma). Low temperature plasma may be formed through the combination of chemical reaction, plasma cleaning and UV radiation. The sterilization chamber may be first evacuated before being filled with the chemical sterilant. Alternatively, the sterilization chamber may be filled with another gas and/or a number of materials could be introduced in sequence to either sensitize or sterilize items in the chamber.

The pervasive coverage of a chemical vapor sterilizer may be beneficial since it may get around mechanical obstructions (other than a mask with a seal or some contact tooling) and may sterilize complex parts, particularly with complex shapes. Additionally, a chemical vapor sterilizer may concurrently sterilize tooling and fixtures used to process the device.

Liquid chemical sterilzation baths may also be considered. This is typically performed by immersing the device in a tank or chamber. In this case, at least one other sterilization protocol may be advantageous if applied to recesses and interior voids either to remedy poor penetration and/or air-locking or to prevent entrapment of fluids and/or reduce drying times.

Chemical sterilization protocols often use one or more chemicals which have a high level of reactivity with surfaces of the product. However, such high levels of reactivity may be damaging to components of the product. For example, organic materials may be degraded, metals may be corroded or tarnished, and fine features may be eroded. Also, there may be reduced penetration of a chemical sterilant into a product with almost enclosed interior details or passages, such that those interior details of the product may not be sterilized to the desired extent.

Heating the device is another example of a protocol which may be used to sterilize the device. Heating the device may be accomplished directly or indirectly. Direct heating may include, for example: baking the device in a heated oven (e.g. medical devices containing electronics may be baked at about 145° C. for 10 minutes without reflowing the solder; devices without electronics may be baked at higher temperatures, such as 160° C. for devices that include polymers such as silicone; devices that include only metal, ceramic and/or glass may be heated to even higher temperatures); scouring a surface of the device with a laser (e.g. a 1064 nm laser); inductive heating using a magnetic field; heating using plasma vapourized hydrogen peroxide; microwaves to heat cellular organisms; or flame sterilizing the device (e.g. heating at 900° C. for 5 seconds). Indirect heating may include, for example, using heated liquids, gases or both, to transfer thermal energy to the device. Steam (e.g. heating at 100° C. for 2 hours) and superheated steam (e.g. heating 125° C. for 20 minutes) are examples of heated gases which may be used to transfer thermal energy to the device.

It would be understood that some sterilization protocols may include characteristics of different classes of sterilization protocols. For example, sterilizing using plasma vapourized hydrogen peroxide results in both hot plasma being discharged as well ionic and emitting UV irradiation. Similarly, microwaves may be considered to be a penetrating radiation from which sensitive electronics may need to be masked, though sterilization occurs through a thermal process in that cellular organisms are heated by the microwave irradiation.

Some sterilization protocols act on surfaces of the device (e.g. chemical sterilants and ionizing radiation, such as UV irradiation), while other sterilization protocols additionally act on the interior of a solid (e.g. penetrative radiation). Portions of a device critical to sterility may include exterior and interior portions that can act as a vector for organisms. For example, critical portions include portions which may communicate organisms to the user directly, or portions of the device from which organisms can migrate during storage, shipping, handling or use to those portions of the device which may communicate the organisms to the user. Non-critical portions of a device are portions which can not act as a vector for organisms. Non-limiting examples of a non-critical portion include an exterior surface of outermost packaging, a fully encapsulated surface (such as those on components that have been over-molded), a surface which is intrinsically sterile, and a portion that is already sterile. An example of a portion that is already sterile includes a component that is encapsulated in a hot over-molding process. Such a process may inject the over-mold material at 160° C. or higher and, consequently, in addition to encapsulating the device the process may also sterilize what is encapsulated as well as the injection tooling. An example of a portion that is intrinsically sterile includes a surface having toxic metals (such as silver). An example of a portion that is already sterile includes a component assembled or encapsulated in a high temperature environment without subsequent exposure to a non-sterile environment.

It will be apparent to one of skill in the art that a sterilized item is “sterilized” when all the critical portions of the device are sterilized, and that it is unnecessary to use sterilization protocols that act on the whole of the interior of the solid portions of the device. That is, it is not necessary to irradiate the whole of the item with penetrative radiation (e.g. X-ray irradiation) or otherwise submit non-critical portions of a device to sterilization in order to consider the item “sterilized”. On the other hand, all critical portions (including critical interior portions) must have been adequately exposed to at least one sterilization protocol for an item to be considered “sterilized”. Further, when more than one sterilization protocol is used to achieve full coverage, it is desirable for the several sterilization protocols to be applied in sufficiently close proximity in time such that organisms do not have the time to migrate from an unsterilized portion to a previously sterilized portion.

### Masks

A mask generally refers to something that prevents a portion of an article from change when the article is subjected to a treatment. For example, a mask may be a physical agent that physically shields or protects a portion of an article from change when the article is subjected to a treatment. Alternatively, a mask may be intrinsic to the nature of the treatment, where the characteristics of the treatment result in a portion of an article not being treated. That is, the characteristics of the treatment result in a portion of the article being masked from the treatment.

For example, masking tape may shield a portion of an item from painting. In another example, paint may be applied with a brush to only one portion of an item. In yet another example, paint may be sprayed on an item for a period of time which results in a portion of the item being unpainted, but where paint spraying for a longer period of time could result in the unpainted portion of the item being painted. In all cases, the unpainted portion of the item is masked from the paint.

In the current application, a mask refers to something that prevents a portion of a device from being sterilized by a sterilization protocol. In some examples, a mask is a physical agent that physically shields a portion of the device from a sterilization protocol. An agent could be considered a mask with respect to one sterilization protocol, but could be considered to not be a mask with respect to another sterilization protocol. A mask may shield the desired portion of the article by transforming one sterilization agent into another sterilization agent, for example by absorbing damaging primary radiation (e.g. gamma radiation) and emitting sterilizing secondary radiation (e.g. X-ray or UV radiation) having reduced damaging effects. Alternatively, a mask may produce a reactive chemical substance or plasma with sterilizing properties when exposed to radiation.

Accordingly, a method that includes “masking a portion of a device from a sterilization protocol using a mask” would be understood to include both masking the device with a physical mask, as well as masking a device using a mask which is intrinsic to the nature of the sterilization protocol. For example, “masking a portion of a device from a sterilization protocol using a mask and sterilizing a portion of the device using the sterilization protocol” would be understood to include any of: (a) applying a physical barrier to shield a portion of the device from a sterilizing agent; (b) selectively sterilizing a portion of the device by using a sterilization protocol that avoids applying a sterilizing agent to a portion of the device; and (c) non-selectively applying a sterilizing agent to the device for a period of time where the sterilizing agent does not sufficiently contact a portion of the device in order to sterilize that portion of the device.

One specific example of a mask is a radiation shield (also known as a shadow mask) which blocks or attenuates the transmission of penetrating radiation, such as gamma radiation. Such a radiation shield could be used to shield an electronic portion of the device from a broad exposure of radiation. The radiation shield may be a simple shape or a complex shape, depending on the nature and location of the portion of the device to be shielded. The radiation shield could be a piece of tooling or fixturing. The radiation shield could be a component attached to or embedded in the device to be sterilized.

Another specific example of a mask for radiation-based sterilization protocols is a device that focuses or narrows the beam of radiation, for example a collimator, a focusing element or a beam scanner. In this case, radiation is selectively applied where wanted rather than blocked from places where not wanted.

Another specific example of a mask is a sealing instrument which blocks the sealed portion of the device from a chemical sterilant. The sealing instrument could be a piece of tooling or fixturing. The sealing instrument could be a component temporarily or semi-permanently attached or affixed to the device to be sterilized. The sealing instrument could be, for example, a cap, breakable seal, label, etc. In methods and systems which use radiation as a sterilization protocol and where the sealing instrument is positioned between the device to be sterilized and the radiation source, it may be desirable for the sealing instrument to be transparent to the radiation.

Specific examples of masks which are intrinsic to the sterilization protocols include: (a) dipping a portion of a device into a sterilizing bath while leaving a portion of the device out of contact with the sterilizing bath, (b) applying a sterilizing chemical gas to the device for a period of time to prevent adequate sterilization of the entire device and leaving a portion of the device untreated by the gas, (c) applying radiation to only a portion of the device. In a method which uses a mask which is intrinsic to the sterilization protocol, the mask is not a physical object and would not, therefore, be able to be removed.

In some situations a single mask could be used as both the mask for a first sterilization protocol and a second sterilization protocol. For example, a steel tube may create a separation where X-rays given off by the tube provide a third means of sterilization that acts on the area occluded by the seal.

It may be observed that a mask may not be a complete protection against the sterilization protocol that it protects against but merely a means of attenuating the effect of that protocol to a safe level for the area masked.

It may also be observed that masks may be incorporated into the device being sterilized in order to provide a masking function in order to protect sensitive components from at least one sterilization protocol.

It may also be observed that certain features of a device may provide a masking function in that they block or obstruct at least one sterilization protocol that may be otherwise effective in sterilizing the device. These features could comprise components that absorb, attenuate and/or dissipate the sterilant and features that prevent sufficient exposure such as convoluted passages, small apertures that limit exposure and blind passages that are capable of producing vapor locks.

### Sterile In-Line Assembly Process

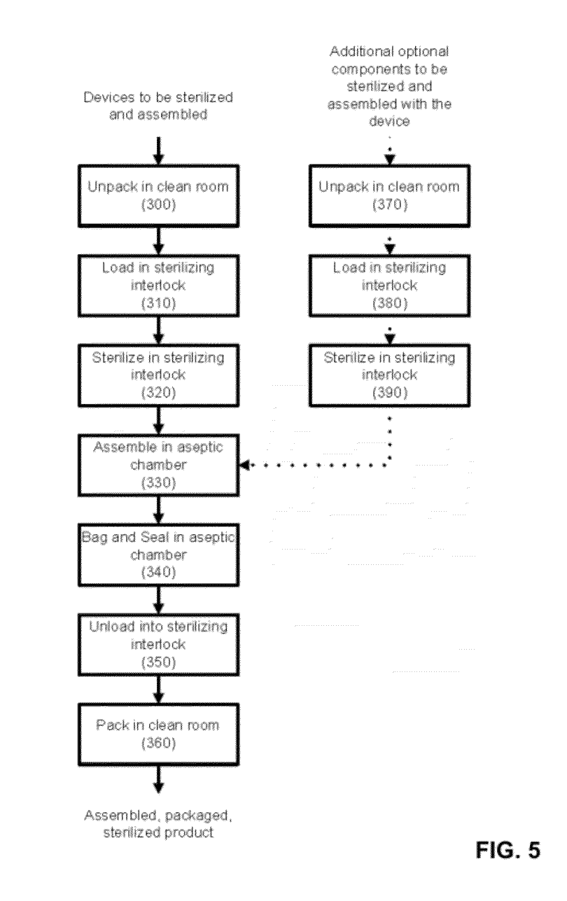

The method for sterilizing a device according to the present application may be applied to a sterile in-line assembly process. Exemplary sterile assembly processes are illustrated in FIG. 5.

As noted above, some aseptic processes, components or subassemblies are input already sterilized and contained in sterile packaging. Outer packaging is removed in a clean environment and then loaded into one or more interlocks. Inner packaging is removed after sterilizing the packaging and the interlock, sterilization may be the hybrid sterilization described above for assemblies or complex parts. Parts are then passed into an aseptic chamber where they are assembled. Risk to the product is low, however there is still residual risk associated with the integrity of packaging and the aseptic environment, particularly any contamination of the assembly tooling. The use of sterile packaging for parts supply and the additional sterilization in the interlock may add considerable cost and complexity.

In a method according to the present disclosure, components or subassemblies which require sterilization according to the present application are unpacked in a clean room 300. It is desirable for the devices to be clean and free from residues which might inhibit sterilization but they may not need to be already sterile. The unpacked devices are loaded into a sterilization interlock 310 and sterilized using a method according to the present application 320. In the event that a complex component or subassembly is being input, the method generally includes: masking a first portion of the device from a first sterilization protocol using a first mask, resulting in a first unmasked portion of the device; sterilizing the first unmasked portion of the device using the first sterilization protocol; and sterilizing the device using a second sterilization protocol. It may still be possible that additional parts are assembled in an aseptic chamber 330 with other sterilized devices or with sterilized components brought into the aseptic chamber, as described below. As introduction of the sterilized components are optional, the associated steps are illustrated using dotted lines. The assembled product is bagged and sealed while in the aseptic chamber 340 and then unloaded into a sterilizing interlock 350. The bagged product is packaged while in a clean room 360.

If the device needs to be assembled with additional components (where those additional components do not include portions of the components which need to be shielded from a sterilization protocol) the components may be sterilized using a single sterilization protocol. In such an exemplary assembly process, the additional components are unpacked in a clean room 370. It is desirable for the components to be clean and free from residues which might inhibit sterilization. The components are loaded into a sterilization interlock 380, and sterilized using a single sterilization protocol 390 (e.g. penetrative radiation or chemical sterilization) without using a mask to shield portions of the components.

Preferably, clean components are assembled first in a clean or sterile environment and then sterilized as a unit. This reduces the number of interlocks needed, possibly to one, or possibly even none if the main chamber is cyclically sterilized. Where the components differ in tolerance to and/or effectiveness of various possible sterilization methods, the options are either the use of a single sterilization protocol which may either not exist or be undesirable or a hybrid sterilization protocol where a plurality of sterilization protocols are selectively applied. Hybrid sterilization facilitates the use of more effective or suitable sterilization protocols for portions of the assembly and/or enables the use of faster or more effective methods. Preferably, as much assembly as possible, preferable all assembly processes are performed prior to sterilization which reduced or eliminates parallel processing paths, chamber interlocks, and aseptic assembly tasks.

The clean room provides a buffer to reduce contamination of devices or components while they are being removed from packaging, and to reduce the possibility that contaminants will enter the interlock. The interlock provides a transitional space between the ambient environment in the clean room and the aseptic environment in the aseptic chamber. Incoming devices and components are prepared and sterilized in the interlock. The interlock may be adapted to reduce the possibility of contamination by various means, such as positive airflow, low pressure or vacuum during transfers. The interlock may be sterilized during each loading or unloading cycle. The aseptic chamber is only exposed to the interlock following a sterilization cycle. The devices are assembled, and optionally bagged and sealed, in the aseptic chamber or the outgoing interlock thereof. The aseptic chamber may receive a single device at a time, or may receive batches of devices. The aseptic chamber may be sterilized using a maintenance program, or may be continually filled with a sterilant on a cyclic basis. In a still more preferable embodiment, sterilization may be done within the same chamber as other operations so that the more pervasive sterilization protocols being used may also serve to maintain the aseptic environment.

The process may include a plurality of interlocks and/or a plurality of aseptic chambers. A plurality of interlocks allows, for example, one interlock to be used for receiving devices to be sterilized from the clean room and another interlock is used for received assembled products from the aseptic chamber. Alternatively, two parallel interlocks could be used alternately in order to balance processing and loading/unloading cycle times.

The sterile in-line assembly process illustrated in FIG. 5 is only one embodiment of possible assembly processes. Sterile in-line assembly processes may have separate interlock and sterilizing chambers. The devices to be sterilized and assembled may be double bagged and unpacking may be performed in a separate environment, such as a glove box. Loading the devices in the sterilizing interlock may be an automated, a semi-automated or manual process.

Sterilizing the devices using a method according to the present application may be a batch process with automated part handling. Sterile parts and tools may be supplied within a container which is removed following sterilization. Sterilization may be preceded by a conditioning process, such as humidification, desiccation, evacuation, and/or rinsing.

With devices which are already assembled and which only require sterilization, the assembly step is unnecessary. Assembled devices may be inspected, tested, and/or marked. Defective or failed products may be rejected and diverted away from the final product.

The sterilized, assembled products may be bagged and sealed in sterile packaging, or may be encapsulated in an alternative manner, such as by placement in a capsule, blister pack, canister, and/or conformal coating.

Packing the assembled, sterilized product may include placing the product in bulk packaging, and may further include cartooning, labeling, and/or inserting.

Small batches of devices or components may be accumulated in the interlock, for example on trays or holding fixtures. Accumulation may be used to balance sterilization time with assembly time. Incoming materials, as well as tools used in the interlock may be sterilized with each cycle. Depending on the materials used in the interlock, and to maintain the aseptic environment, this chamber may be fitted with a gas recycling system for continuous chemical sterilization. It is desirable that tools used in the aseptic chamber be compatible with a sterilization process. For example, VHP (Vapor Hydrogen Peroxide) robots or chemically resistant vacuum robots may be used.

One example of a device which could be sterilized according to the present description is a syringe and needle. Some portions of the syringe could be sterilized at least by one protocol and other portions at least by another protocol. For example, the sharp metal needle could be protected both mechanically and sterilely by a removable cap. This removable cap could mask a chemical sterilization protocol that would otherwise react with the metal needle. The surfaces of the syringe and cap could be simultaneously sterilized by one or more penetrating radiation protocols. The penetrating radiation protocols and the metal of the needle may additionally produce hard secondary radiation in the interior of the metal tube.

Embodiments herein are intended to provide improved systems and methods that may accomplish sterilization of complex devices with reduced constraints due to component sensitivity and/or with increased efficacy by the use of the most appropriate methods. Further, the embodiments are intended to provide a means of applying additional sterilization to critical components and subassemblies.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

Embodiments of the disclosure may be represented as a computer program product stored in a machine-readable medium (also referred to as a computer-readable medium, a processor-readable medium, or a computer usable medium having a computer-readable program code embodied therein). The machine-readable medium may be any suitable tangible, non-transitory medium, including magnetic, optical, or electrical storage medium including a diskette, compact disk read only memory (CD-ROM), memory device (volatile or non-volatile), or similar storage mechanism. The machine-readable medium may contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations may also be stored on the machine-readable medium. The instructions stored on the machine-readable medium may be executed by a processor or other suitable processing device, and may interface with circuitry to perform the described tasks.

The above-described embodiments are intended to be examples only. Alterations, modifications and variations may be effected to the particular embodiments by those of skill in the art.

What is claimed is:

1. A method for sterilizing a device, the method comprising:
masking a portion of the device from a sterilization protocol using a mask;
sterilizing a portion of the device using a first sterilization protocol; and
sterilizing a portion of the device using a second sterilization protocol;
wherein the mask shields the masked portion of the device from at least one of the sterilization protocols or attenuates the effect of at least one of the sterilization protocols in the masked portion.

2. The method according to claim 1, wherein the mask alters the first sterilization protocol, the second sterilization protocol, or both the first and the second sterilization protocols so that shielding the masked portion of the device results in exposure of the masked portion to an altered sterilization protocol.

3. A method for sterilizing at least a portion of a device, the method comprising:
masking a first portion of the device from a first sterilization protocol using a first mask, resulting in a first unmasked portion of the device;
sterilizing the first unmasked portion of the device using the first sterilization protocol; and
sterilizing the device using a second sterilization protocol.

4. The method according to claim 3, further comprising:
masking a second portion of the device from a second sterilization protocol using a second mask, resulting in a second unmasked portion of the device;
wherein the step of sterilizing the device using a second sterilization protocol comprises sterilizing the second unmasked portion of the device using the second sterilization protocol.

5. The method according to claim 3, wherein the first mask alters the first sterilization protocol, the second sterilization protocol, or both the first and the second sterilization protocols so that shielding the masked portion of the device results in exposure of the masked portion to an altered sterilization protocol.

6. The method according to claim 4, wherein the first mask, the second mask, or both the first and second masks, alters the first sterilization protocol, the second sterilization protocol, or both the first and the second sterilization protocols so that shielding the masked portion of the device results in exposure of the masked portion to an altered sterilization protocol.

7. The method according to claim 6 wherein the first mask, the second mask, or both the first and the second mask may be an item introduced into the process, or may inherently be a portion of the device.

8. The method according to claim 1, further comprising removing at least one mask from the device.

9. The method according to claim 1, wherein the first sterilizing protocol and the second sterilizing protocol are applied sequentially or concurrently.

10. The method according to claim 1, wherein one or more portions of the device are exposed to both the first and the second sterilization protocols.

11. The method according to claim 1, further comprising masking a portion of the device with a further mask and sterilizing the unmasked portion of the device using a further sterilization protocol.

12. A system for sterilizing a device, the system comprising:
a first sterilant;
a second sterilant; and
a mask for shielding a portion of the device from at least one of the sterilants.

13. The system according to claim 12, wherein the mask is a component attached to or embedded in the device to be sterilized.

14. The system according to claim 12, wherein the mask is a component that is removed when the device is used.

15. The system according to claim 12, further comprising another mask for shielding a portion of the device from the other of the first and second sterilants, or from a further sterilant.
